(19) United States
(12) Patent Application Publication
(10) Pub. No.: US 2022/0022955 A1
(43) Pub. Date: Jan. 27, 2022

(54) SYSTEMS AND METHODS FOR ABLATION MONITORING

(71) Applicant: THE TRUSTEES OF COLUMBIA UNIVERSITY IN THE CITY OF NEW YORK, New York, NY (US)

(72) Inventors: Elisa E. KONOFAGOU, New York, NY (US); Vincent SAYSENG, Long Island City, NY (US); Elaine WAN, Fresh Meadows, NY (US)

(73) Assignee: THE TRUSTEES OF COLUMBIA UNIVERSITY IN THE CITY OF NEW YORK, New York, NY (US)

(21) Appl. No.: 17/493,108

(22) Filed: Oct. 4, 2021

Related U.S. Application Data
(63) Continuation of application No. PCT/US2020/026888, filed on Apr. 6, 2020.
(60) Provisional application No. 62/959,560, filed on Jan. 10, 2020.
(60) Provisional application No. 62/935,437, filed on Nov. 14, 2019.
(60) Provisional application No. 62/829,499, filed on Apr. 4, 2019.

(51) Int. Cl. A61B 18/14; A61B 8/08; A61B 8/12; G16H 40/63; G16H 30/40

(52) U.S. Cl. CPC A61B 18/1492; A61B 8/0883; A61B 2018/00357; G16H 40/63; G16H 30/40; A61B 8/12

(57) ABSTRACT

System and methods for monitoring cardiac ablation procedures are disclosed. The system can comprise an imaging device and an image processor. The imaging device can be configured to acquire successive frames and radio frequency signal data of a heart. The image process, coupled to the imaging device, can be configured to obtain a signal envelope of the radio frequency signal data, generate a strain map based on the signal envelope, apply a strain threshold to the strain map for classification of lesion tissue, and provide an image which visualizes lesion formation during the cardiac ablation procedures in real-time.

### CROSS-REFERENCE TO RELATED APPLICATION

This application is a continuation of International Patent Application No. PCT/US 2020/026888 filed Apr. 6, 2020, which claims priority to U.S. Provisional Patent Applications Nos. 62/829,499, which was filed on Apr. 4, 2019, 62/935,437, which was filed on Nov. 14, 2019, and 62/959,560, which was filed on Jan. 10, 2020, the entire contents of which are incorporated by reference herein.

### STATEMENT REGARDING FEDERALLY SPONSORED RESEARCH

This invention was made with government support under EB006042 and HL096094 awarded by the National Institutes of Health. The government has certain rights in the invention.

### BACKGROUND

Cardiac arrhythmia can be characterized by abnormal electrical activation in the heart. One known therapy for cardiac arrhythmia is catheter ablation. Thin, tubular, flexible instruments (e.g., catheters) can access the heart via the major blood vessels. Specialized catheters can deliver energy to ablate specific areas of the heart for creating scars or lesions. Multiple lesions can be formed and can present a barrier of non-conductive tissue that effectively breaks the electrical circuit causing the abnormal rhythm.

Certain ablations can be unsuccessful and can necessitate additional ablation procedures. The placement and size of the lesions can be factors affecting the success of an ablation procedure. If lesions are placed too far apart, allowing gaps to form, or are of insufficient depth (e.g., non-transmural), the electrical circuit causing the arrhythmia can reconnect, allowing the arrhythmia to return. Forming lesions close enough and deep enough to permanently terminate the arrhythmia can be challenging, at least in part because certain techniques to visualize the lesions within the tissue being ablated can be unreliable.

Thus, there is a need for improved systems and methods for imaging a tissue during ablation to identify and assess structures, such as lesions formed during ablation.

### SUMMARY

The disclosed subject matter provides systems and methods for monitoring cardiac ablation procedures. An example system includes an imaging device configured to acquire successive frames and radio frequency signal data of a heart and an imaging processor coupled to the imaging device. The imaging processor can be configured to obtain a signal envelope of the radio frequency signal data, generate a strain map based on the signal envelope, apply a strain threshold to the strain map for classification of lesion tissue, and provide an image which visualizes lesion formation during the cardiac ablation procedures in real-time. In some embodiments, the imaging device can further include a catheter for cardiac ablation procedures. In non-limiting embodiments, the catheter can be used for real-time imaging through intracardiac echocardiology.

In certain embodiments, the radio frequency signal data can be obtained using an acquisition frame rate less than 500 frames per second (fps). The strain map can be generated based on axial, incremental, and/or cumulative axial strain of the successive frames. The axial, incremental, and cumulative axial strain can be estimated using the signal envelope. In some embodiments, the axial strain can be estimated based on axial, incremental, and/or cumulative displacement of the heart. The axial, incremental, and/or cumulative displacement can be estimated by performing a cross-correlation on the signal envelope. In certain embodiments, a lateral, incremental lateral, or cumulative lateral strain can be estimated based on lateral, incremental, and/or cumulative lateral displacement of the successive frames. In non-limiting embodiments, the lesion formation can be represented as having a near-zero or relatively lower magnitude strain.

An example method for monitoring cardiac ablation procedures can include obtaining a signal envelope of a radio frequency signal data, generating a strain map based on the signal envelope, applying a strain threshold to the strain map for classification of lesion tissue, and providing an image which visualizes lesion formation during the cardiac ablation procedures in real-time. In non-limiting embodiments, the method can further include performing a cross-correlation on the signal envelope. In some embodiments, the radio frequency signal data can be obtained using an acquisition frame rate of less than 500 frames per second. In certain embodiments, the method can further include estimating a lateral, incremental lateral, or cumulative lateral strain based on lateral, incremental, and/or cumulative lateral displacement of the successive frames.

In non-limiting embodiments, the strain map can be generated based on axial, incremental, and/or cumulative axial strain of the successive frames. The axial, incremental, and cumulative axial strain can be estimated using the signal envelope. In some embodiments, the axial strain can be estimated based on axial, incremental, and/or cumulative displacement of the heart. The axial, incremental, and/or cumulative displacement can be estimated by performing a cross-correlation on the signal envelope. In non-limiting embodiments, the lesion formation can be represented as having near-zero magnitude strain.

### BRIEF DESCRIPTION OF THE DRAWINGS

FIG. 1 is a block diagram illustrating one or more elements of the presently disclosed system.

FIG. 2 is a flow diagram of exemplary methods of the presently disclosed subject matter.

FIGS. 3A-3D provide images showing an exemplary coregistration procedure in accordance with the disclosed subject matter. FIG. 3A provides a photograph showing lesion lines excised from the myocardium. FIG. 3B provides a translated image using anatomical markets. FIG. 3C provides an ultrasound image coregistered with a B-mode image. FIG. 3D provides an ultrasound image showing a binary mask that indicates the lesion area.

FIGS. 4A-4D provide images showing strain-based lesion maps. FIG. 4A provides an ultrasound image showing unablated myocardium. FIG. 4B provides an ultrasound image showing ablated tissue. FIG. 4C provides an ultrasound image showing a first lesion, a second lesion, and a gap between the lesions. FIG. 4D provides an ultrasound image showing a first lesion, a second lesion, a third lesion, and gaps between the lesions.

FIGS. 5A-5H provide post-ablation IME lesion maps compared to gross pathology. Three different lesion lines, including three lesions and two gaps in the canine left ventricle (LV) are shown (FIGS. 5A-5F). One lesion generated in the canine right ventricle (RV) is shown (FIGS. 5G-5H).

FIG. 6 provides a graph showing the Dice Similarity Coefficient (DSC) in accordance with the disclosed subject matter.

FIGS. 7A-7H provide images showing thresholded strain-based lesion maps compared to gross pathology. Strains for lesions and gaps are determined against the ground truth areas for a first LV lesion line (FIGS. 7A and 7B), a second LV lesion line (FIGS. 7C and 7D), a third LV lesion line (FIGS. 7E and 7F), and an RV lesion (FIGS. 7G and 7H).

FIGS. 8A-8C provide ultrasound images showing ablation of the cavotricuspid isthmus (CTI) in the RA of a patient with atrial flutter. FIG. 8A provides an ultrasound image showing a baseline of CTI. FIG. 8B provides an ultrasound image showing a first lesion generated proximal to a tricuspid valve. FIG. 8C provides an ultrasound image showing the CTI at the end of the ablation procedure.

FIG. 9 provides a graph showing median strains where CTI decreases after ablation.

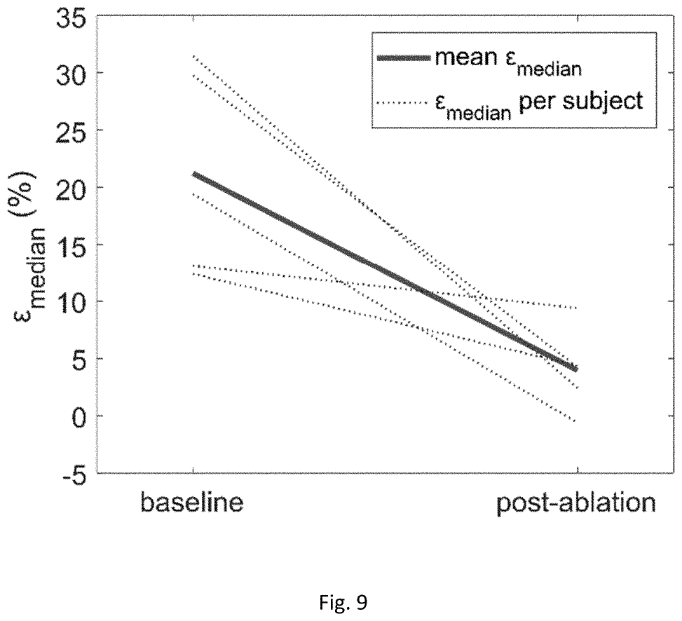

### DETAILED DESCRIPTION

The disclosed subject matter provides techniques for monitoring cardiac ablation procedures. The disclosed subject matter can image a heart in real-time and visualize lesion tissue by estimating the strain of the heart. The disclosed subject matter can reduce the number of repeated catheter ablations for arrhythmia patients by allowing clinicians to visualize lesion formation during the ablation procedure.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used here, the term “coupled” refers to the connection of a system component to another system component by a suitable connection technique known in the art. The type of coupling used to connect two or more system components can depend on the scale and operability of the system. For example, and not by way of limitation, the coupling of two or more components of a system can include connecting the imaging device to the imaging processor via a wired connection and/or a wireless connection.

A “subject” herein can be a human or a non-human animal, for example, but not by limitation, rodents such as mice, rats, hamsters, and guinea pigs; rabbits; dogs; cats; sheep; pigs; goats; cattle; horses; and non-human primates such as apes and monkeys, etc.

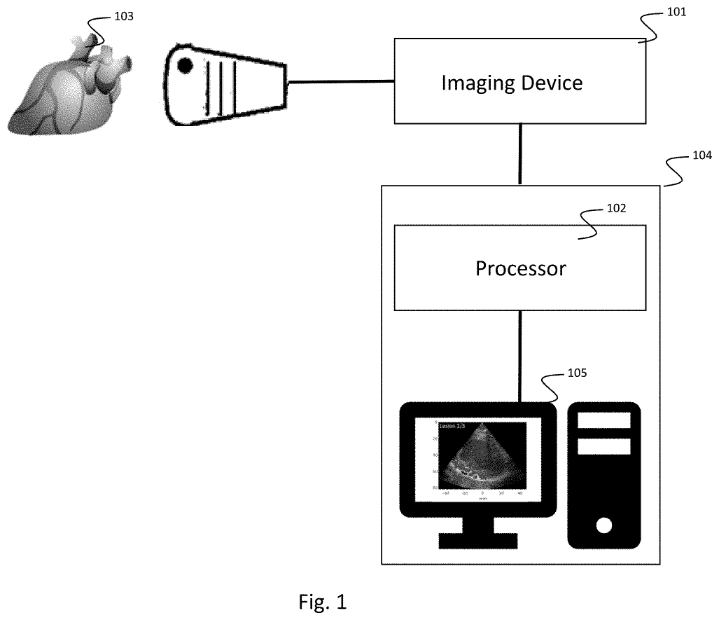

An example system for monitoring cardiac ablation procedures can include an imaging device 101 and an image processor 102. For example, as shown in FIG. 1, the imaging device 101 can be an ultrasound device configured to be coupled to the imaging processor 102 and obtain ultrasound data. The imaging processor 102 can be an electronic circuit that can be configured to perform various operations on the ultrasound data.

In certain embodiments, the imaging device can be configured to acquire successive frames and radio frequency signal data of a target tissue 103. For example, a target tissue with a lesion can be imaged by the imaging device 102. In non-limiting embodiments, the imaging device can utilize a transmit sequence and/or a compounded sequence for imaging the target tissue, which can be customized based on a subject. The transmit sequence refers to the process by which ultrasound pulses can be transmitted into the tissue. For example, not by way of limitation, the transmit sequence can include a sequence for plane wave imaging, diverging wave imaging, compounding, wide beam, multi-line transmit, and multi-focus imaging. In non-limiting embodiments, the disclosed system can provide high temporal resolution utilizing a high-frame-rate transmit sequence (e.g., a transmit sequence with a frame rate >100 Hz). For example, where a subject has epicardial lesions, the transmit sequence can include at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 35, at least about 40, at least about 45, at least about 50, at least about 60, at least about 70, at least about 80, at least about 90, at least about 100 steered plane waves. In non-limiting embodiments, the transmit sequence can include a virtual source which can be located greater than about 1 mm, about 10 mm, about 20 mm, about 30 mm, about 40 mm, about 50 mm, about 100 mm, about 150 mm, about 200 mm, about 250 mm, about 300 mm, about 350 mm, about 400 mm, about 450 mm, about 500 mm, about 550 mm, about 600 mm, about 650 mm, about 700 mm, about 750 mm, about 800 mm, about 850 mm, about 900 mm, about 950 mm, or about 1000 mm. In non-limiting embodiments, the imaging device can include a probe (e.g., transducer) to generate the transmit sequence at a frame rate of about 100 frames per second (fps), about 150 fps, about 200 fps, about 250 fps, about 300 fps, about 350 fps, about 400 fps, about 450 fps, about 500 fps, about 600 fps, about 650 fps, about 700 fps, about 750 fps, about 800 fps, about 850 fps, about 900 fps, about 950 fps, or about 1000 fps. In non-limiting embodiments, the frame rate of the transmit sequence can be at least 100 Hz. In non-limiting embodiments, the transmit sequence can include about 30 virtual sources, about 20 virtual sources, about 15 virtual sources, about 10 virtual sources, about 5 virtual sources, or about 1 virtual source. In non-limiting embodiments, the angular aperture of the field-of-view for the transmit sequence can be about 180°, about 150°, about 90°, about 70°, about 40°, about 25°, or about 10°. In non-limiting embodiments, the angular aperture of the field-of-view can be up to about 150°.

In non-limiting embodiments, a customized transmit sequence (e.g., 1.5 seconds of focused imaging at 30 fps, followed by 1 second of a single diverging wave sequence with 6.5 mm virtual source and 600 fps frame rate) can be used for imaging a subject with atrial flutter. The obtained imaging data using the customized transmit sequence can be used to estimate displacement and strain, and the former to provide a B-mode reference frame.

In non-limiting embodiments, the imaging device can generate a compounded sequence for imaging a target tissue with a lesion. The compounded sequence can be a sequence that can compound a type of transmit sequence that aggregates unfocused (e.g., diverging waves or plane waves) ultrasound transmits emitted from different virtual sources. The compounded sequence can include various ranges of virtual sources, focus level, frame rate, depth level, and or angular aperture. For example, the compounded sequence can include about 30 virtual sources, about 20 virtual sources, about 15 virtual sources, about 10 virtual sources, about 5 virtual sources, or about 1 virtual source. The focus level can be greater than about 1 millimeter (mm), about 5 mm, about 10 mm, about 25 mm, about 50 mm, about 100 mm, about 250 mm, about 500 mm, or about 1000 mm. The frame rate can be less than about 1000 fps, about 950 fps, about 900 fps, about 850 fps, about 800 fps, about 750 fps, about 700 fps, about 650 fps, about 600 fps, about 550 fps, about 500 fps, about 450 fps, about 300 fps, about 250 fps, about 200 fps, about 150 fps, about 100 fps, about 50 fps or about 10 fps. The focus level can be greater than about −1 mm, about −5 mm, about −10 mm, about −25 mm, about −50 mm, about −100 mm, about −250 mm, about −500 mm, or about −1000 mm. In non-limiting embodiments, the virtual source can be any distance behind the array. In certain embodiments, when the compounded sequence is used, the “focus” can refer to the distance between the virtual source and the array. In non-limiting embodiments, the focus of diverging wave sequences can be behind of the ultrasound array (e.g., negative focus value). For example, a focus of −20 mm can be equivalent to the focus of about 20 mm behind the array. The angular aperture can be about 180°, about 90°, about 70°, about 40°, about 25°, or about 10°. In non-limiting embodiments, a high-frame-rate compounded sequence (e.g., 15 virtual sources, focus located 21 mm behind the transducer, 460 fps, depth 80 mm, angular aperture 90°) can be employed to obtain an ultrasound data of the target tissue.

In certain embodiments, the imaging device can include a probe 105. For example, the probe can be a phased array. In non-limiting embodiments, the probe can include any intracardiac echocardiography catheters. In certain embodiments, 3D intracardiac catheters can be used. In non-limiting embodiments, the imaging device can be configured to perform intracardiac myocardial elastography (IME) imaging. IME is a strain-based lesion mapping technique. For example, and not by way of limitation, the probe can be a catheter which can be configured to obtain radio frequency signal data. In non-limiting embodiments, the catheter can be an ultrasound catheter for intracardiac echocardiography (ICE). The ICE catheter can be used to monitor a target tissue during ablation and/or lesion formation. For example, the ICE catheter can utilize a high-frame-rate acquisition sequence to estimate the strain of a heart (e.g., the atrial or ventricular walls). The disclosed IME technique can be a platform-independent technique that can be programmed onto ICE systems. The IME technique can be integrated into an ablation monitoring process without requiring specialized hardware for mapping a lesion. In non-limiting embodiments, the ICE catheter can be utilized to obtain the successive frames and radio frequency signal data over the duration of the systole of a target heart tissue. In non-limiting embodiments, the radio frequency data can be collected over any phase of the cardiac cycle. For example, the strain can be measured and accumulated during diastole and/or systole.

In certain embodiments, the radio frequency data and image frames can be obtained using the imaging device with various acquisition frame rates. For example, the radio frequency can be obtained using an acquisition frame rate less than about 1000 fps, about 950 fps, about 900 fps, about 850 fps, about 800 fps, about 750 fps, about 700 fps, about 650 fps, about 600 fps, about 550 fps, about 500 fps, about 450 fps, about 300 fps, about 250 fps, about 200 fps, about 150 fps, or about 100 fps. In non-limiting embodiments, the acquisition frame rate can be at least 100 Hz.

In certain embodiments, the image processor 102 can be coupled to the imaging device 101 and configured to perform processing procedures on the acquired frames and radio frequency data. For example, the image processor can be an electronic circuit which can perform various analyses on the acquired frames and radiofrequency data. The image processor can be included in a computing device 104, which can be connected to the imaging device 101 (e.g., ultrasound device).

In certain embodiments, the image processor can obtain radio frequency signal data of target tissue 103 from the imaging device 101. In non-limiting embodiments, the image processor can be configured to obtain a signal envelope based on the radio frequency signal data. For example, raw RF data can be obtained with the imaging device. The imaging processor can beamform the raw RF data by using delay-and-sum. Delay and sum (DAS) is an ultrasound beamforming process by which the ultrasound beam can be shaped. DAS can apply time delays to create an interpretable image. In certain embodiments, any beamforming process can be used in the disclosed system. Axial displacements can be estimated from beamformed RF signals using a 1-D normalized cross-correlation kernel. This can also be expanded to 2D or 3D displacements using 1D or 2D kernels in 2D or 3D search. To process RF data with a lower achievable acquisition frame rate (e.g., less than about 250 Hz), axial displacements can be estimated from the beamformed envelope signals obtained from the RF data. The Hilbert transform can be applied to the radiofrequency data in order to obtain the signal envelope. If the raw data is in in-phase and quadrature components (IQ data), the envelope can be calculated by taking the square root of the sum of squared components.

In certain embodiments, the obtained signal envelope can be used for estimating axial, incremental, and/or cumulative axial displacement of the successive frames. For example, 1D cross-correlation can be applied to the envelope of the radio frequency signal to estimate the axial displacement. In non-limiting embodiments, the disclosed image processor can estimate the axial, incremental, and/or cumulative axial displacement of the successive frames using the radio frequency signal. The incremental displacement can be obtained from the axial displacement. The incremental displacements can be accumulated to estimate the cumulative axial displacement. In non-limiting embodiments, the disclosed image processor can estimate lateral, incremental lateral, and/or cumulative lateral displacement of the successive frames using the radio frequency data.

In certain embodiments, the disclosed image processor can be configured to generate a strain map based on the signal envelope. The disclosed system can estimate axial, incremental, and/or cumulative axial strain based on the axial, incremental, and/or cumulative axial displacement obtained from the signal envelope. For example, the cumulative axial strain can be estimated by calculating the axial spatial gradient of the axial cumulative displacement using a least-squares estimator (e.g., 1D least-squares estimator). In non-limiting embodiments, the disclosed image processor can estimate a lateral, incremental lateral, and/or cumulative lateral strain based on the lateral, incremental lateral, and/or cumulative lateral displacement of the successive frames. The disclosed image processor can generate the strain map by overlaying the estimated axial, incremental, and/or cumulative axial strain on the B-mode.

In certain embodiments, the strain images can be validated against gross pathology. For example, the target tissue can be stained with tetrazolium chloride (TTC). The images of the TTC-stained tissue with lesion lines can be converted to grayscale and aligned with ICE to overlaid onto the B-mode image based on anatomical landmarks. The lesions can be masked by a combination of brightness thresholding and manual segmentation. For example, intensity thresholding and manual segmentation can be employed to create a binary mask that indicated the lesion area by gross pathology.

In certain embodiments, the disclosed imaging processor can adjust a dynamic range of strain image to maintain high image contrast between the lesion and non-lesion part of the target tissue. For example, due to the deformation of the myocardium during the cardiac cycle, a lesion line can move out of the field-of-view at certain time points during systole. Thus, the number of displacement frames accumulated can be different for each acquisition. The strain magnitude can be dependent on the number of displacement frames accumulated. The disclosed imaging processor can provide improved consistency by adjusting the dynamic range of the strain images to provide an improved image contrast between lesion and non-lesion. For example, and not by way of limitation, the upper bound of the dynamic range (DRupper) can be empirically chosen to be half of the median strain at baseline at the number of frames accumulated:

$$D {R}_{upper} = \frac{median ( {ɛ}_{baseline} ( n ) )}{2} \qquad ( 1 )$$

where ε_{baseline}(n) is the masked strain values in the n^{th} accumulated frame at baseline. The lower bound of the dynamic range can be 0%.

Lesion area estimated by IME can be calculated as follows:

$${A}_{s t r a i n} ( mm ) = {A}_{p ixel} * {\sum }_{i = 1}^{N} \{ \begin{matrix}0 & if ɛ ( i ) \geq {ɛ}_{t h r e s h} \\ 1 & if ɛ ( i ) < {ɛ}_{t h r e s h}\end{matrix} \qquad ( 2 )$$

where ε(i) represents strain at a given pixel i within a masked region consisting of N pixels. εₜₕᵣₑₛₕ is the strain threshold, and Aₚᵢₓₑₗ the area of each pixel in mm. Masks can be manually delineated to isolate lesions and gaps. The boundary of the gap masks can be determined by a vector spanning the apex of the two lesions, at the points closest to the endocardial wall.

The absolute (δA) and the relative difference between Aₛₜᵣₐᵢₙ and the areas reported by gross pathology (A_{gross}) can be calculated, given

$${A}_{g r o s s} ( mm ) = {A}_{p ixel} * {\sum }_{i = 1}^{N} \{ \begin{matrix}0 & if pixel ( i ) \geq {pixel}_{t h r e s h} \\ 1 & if pixel ( i ) < {pixel}_{t h r e s h}\end{matrix} , \qquad ( 3 )$$

where the brightness threshold pixelₜₕᵣₑₛₕ can be determined empirically based on the qualitative assessment of the gross pathology images. Aₚᵢₓₑₗ is the area of each pixel in mm. Manual segmentation can be performed to isolate individual lesion areas.

In certain embodiments, the disclosed image process can be configured to apply the strain threshold to the strain map for the classification of lesion tissue. The disclosed system can generate a peak mean Dice similarity coefficient curve (DSC) to determine the threshold for showing a region of tissue classified as a scar (i.e., lesion). For example, the threshold under which a point can consider a lesion, εₜₕᵣₑₛₕ, can be defined as,

εₜₕᵣₑₛₕ=α*median(ε_{baseline}(n)) (4)

In the above, α can be within the range of [0, 1.5]. The optimal εₜₕᵣₑₛₕ can be determined by finding the mean DSC curve across lesion lines, and selecting the a value that yields the highest εₜₕᵣₑₛₕ. The DSC between two binary sets A and B can be determined as,

$$dice ( A , B ) = \frac{2 T P}{2 T P + F P + F N} \qquad ( 5 )$$

Where TP is true positives, FP is false positives, and FN is false negatives. The Dice similarity coefficient (DSC) can range from 0 to 1, and measures the similarity between two binary sets. For example, a score of 1 indicates that the binary masks are identical, while a score of 0 indicates that there is no intersection between the positive values in the binary masks. In non-limiting embodiments, at the decided threshold, the individual lesions and gaps between them can be delineated.

In certain embodiments, the disclosed image processor can be configured to be coupled to an output device 105 and provide an image that visualizes lesion formation. Lesions can be shown as a near-zero magnitude or a relatively lower strain. For example, regions, where strain is below about 30% of the median strain at baseline, can be considered scar/legion. In non-limiting embodiments, the scar lesion can have strain below about 27%, about 25%, about 20%, about 15%, about 10%, about 5%, or about 1% of median strain at baseline. In certain embodiments, the cumulative strain of the lesion can be determined based on the number of frames accumulated. Lesions can be identified by the contrast in the strains between the lesion and non-ablated tissue.

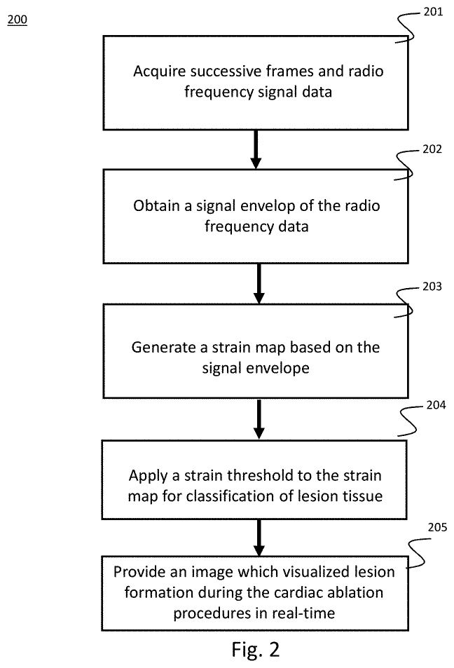

In certain embodiments, the disclosed subject matter can provide methods for monitoring cardiac ablation procedures. As shown in FIG. 2, an exemplary method 200 can include acquiring successive frames and radio frequency signal data from target tissue 201. The successive frames and radio frequency signal data can be obtained using an imaging device that can produce the customized transmit sequences, compounded sequence, and/or acquisition sequences.

In certain embodiments, the method can include obtaining a signal envelope based on the obtained radio frequency signal data 202. The obtained signal envelope can be used for estimating axial, incremental, and/or cumulative axial displacement of the successive frames. For example, 1D cross-correlation can be applied to the envelope of the radio frequency signal to estimate the axial displacement. In non-limiting embodiments, the method further can include estimating the axial, incremental, and/or cumulative axial displacement of the successive frames using the radio frequency signal. The incremental displacement can be obtained from the axial displacement. The incremental displacements can be accumulated to estimate the cumulative axial displacement.

In certain embodiments, the method can further include generating a strain map based on the signal envelope 203. Axial, incremental, and/or cumulative axial strain can be calculated based on the axial, incremental, and/or cumulative axial displacement obtained from the signal envelop. For example, the cumulative axial strain can be estimated by calculating the axial spatial gradient of the axial cumulative displacement using a least-squares estimator (e.g., 1D least-squares estimator). In non-limiting embodiments, the method can further include estimating a lateral, incremental lateral, or cumulative lateral strain based on lateral, incremental lateral, and/cumulative lateral displacement of the successive frames. The disclosed image processor can generate the strain map by overlaying the estimated axial, incremental, and/or cumulative axial strain on the B-mode.

In certain embodiments, the method can further include applying a strain threshold to the strain map for the classification of lesion tissue 204. The disclosed system can generate a peak mean DSC to determine the threshold for showing a region of tissue classified as a scar. The DSC and threshold can be determined using Eqs. 4 and 5.

In certain embodiments, the method can further include providing an image that visualizes lesion formation 205. Lesions can be shown as near-zero or relatively lower magnitude strain. For example, regions, where strain is below about 30% of the median strain at baseline, can be considered scar/legion. In non-limiting embodiments, the scar lesion can have strain below about 27%, about 25%, about 20%, about 15%, about 10%, about 5%, or about 1% of median strain at baseline.

### EXAMPLES

The following Examples are offered to illustrate the disclosed subject matter but are not to be construed as limiting the scope thereof.

### Example 1: Catheter Ablation Lesion Visualization with Intracardiac Strain Imaging in Canines and Humans

The presently disclosed subject matter will be better understood by reference to the following Example. The Example provided as merely illustrative of the disclosed methods and systems, and should not be considered as a limitation in any way.

Among other features, the example illustrates catheter ablation monitoring with intracardiac strain imaging in canines and humans.

Ultrasound-based lesion mapping methods can be used for obtaining real-time feedback during ablations. Unlike MRI-based lesion mapping techniques, which require extensive adjustments in the procedure workflow due to the required additional hardware and technicians, the disclosed technique can be used without extensive adjustments during ablations. For example, the disclosed ablation catheter with near field ultrasound imaging capabilities can provide real-time feedback regarding lesion formation.

The disclosed subject matter provides both methods to identify lesion gaps in open-chest canine models, and methods concerning the diagnostic utility in a clinical setting. Lesion gap resolution was assessed by creating lesion lines comprised of three epicardial lesions and two gaps in three canine left ventricles (LV). In patients, a customized Intracardiac Myocardial Elastography (IME) technique was used to monitor ablation in the cavotricuspid isthmus (CTI) throughout an atrial flutter procedure in five subjects.

Lateral thoracotomy was performed on anesthetized mongrel canines (n=3, 100% male, 26±2.1 kg) to expose the myocardium for epicardial ablation. Due to its cellular, functional, and physiological similarities with the human heart, the canine heart is one of the most popular large animal models in cardiac research. Furthermore, since mongrel canines are genetically diverse, they are close representations of the non-homogenous genetic background of humans. The intracardiac ultrasound catheter (CartoSoundstar, Biosense Webster, Irvine, Calif., USA) was introduced via the external jugular vein and advanced through the superior vena cava. Positioning the probe in the RV provided images of the anterior and anterolateral segments of the LV. Imaging of the RV was performed by positioning the probe in the LV via apical puncture. Epicardial lesions were created in the LV and RV by catheter ablation (Carto 3 System, Biosense Webster, Irvine, Calif., USA). In the LV, a lesion line consisting of three lesions with two gaps were generated in three animals. In the RV, one lesion was created in one animal.

Images were acquired prior to and after each lesion (SoundStar 10F Catheter, Biosense Webster, Irvine, Calif., USA). For the LV lesion lines, images were acquired at baseline and then after each lesion for a total of four time points. For the RV lesion, images were acquired before and after the ablation. Lesions were imaged with one of two ultrasound platforms (the same catheter, Soundstar, was employed). Two canines (two LV lesions lines and one RV lesion) were imaged with an Acuson SC2000 (Siemens, Munich, Germany). The transmit sequence consisted of 24 steered plane waves (virtual source located >300 mm behind the transducer) at a frame rate of about 200-250 fps and depth of 80 mm. The angular aperture of the field-of-view was 70°. One canine (one LV lesion line) was imaged with a Verasonics Vantage (WA, USA) and a modified Acuson Swiftlink Connector (TransducerWorks, PA, USA).

A high-frame-rate compounded sequence was employed (in this example, 15 virtual sources, focus located 21 mm behind the transducer, 460 fps, depth 80 mm, and angular aperture 90°). At the conclusion of the procedure, the myocardium was excised. The lesion line was segmented and placed in the freezer (−18° C.) for 40-60 minutes. The sample was sliced transmurally along the axis of the lesion line. Sections were submerged in 1% tetrazolium chloride (TTC) and placed in an incubator (37°) for at least 40 minutes. TTC stained the lesions white. Photos of the sections with scale bar for reference were obtained (Nikon EOS Rebel T3i, Tokyo, Japan).

Patients were informed of the study's risk prior to obtaining consent. The transmit sequence complied with U.S. Food and Drug Administration (FDA) limits, as of the time of filing this disclosure, on acoustic output. Patients with certain cavotricuspid isthmus (CTI) atrial flutter (n=5, men=60%, age=67±16 years old) underwent RF ablation of the cavotricuspid isthmus of the right atrium (RA), which was imaged with a well-known ICE clinical machine (ViewMate ICE Catheter and Viewmate Z, Abbott, Chicago, Ill.). The ICE catheter was positioned in the RA.

A customized transmit sequence was implemented: 1.5 s of conventionally focused imaging at 30 fps, followed by 1 second of a single diverging wave sequence (−6.5 mm virtual source, 600 fps frame rate). The latter acquisition was used to estimate myocardial displacement and strain; the former was used to provide a B-mode reference frame of end-systole over which to overlay the strain. The ICE ultrasound field-of-view was set to the CTI region proximal to the tricuspid valve. Images were acquired prior, during, and after the CTI ablation procedure. The field-of-view was updated throughout the procedure to ensure that the ablation catheter was in view before, during, and after each lesion. The ablation procedure was considered complete once the achievement of the block was confirmed via coronary sinus pacing.

The different ultrasound platforms were used (e.g., Siemens Acuson, Verasonics Vantage, and Abbott Viewmate Z) for IME imaging. Each hardware platform possessed different parameters. The Acuson provides for coregistering the lesion line and the ICE view through the CARTOSOUND software (Carto 3 System, Biosense Webster, Irvine, Calif., USA), which can mark the position of the ablation catheter in real-time when it is in a plane with the ICE catheter. The Vantage's open programmability allows for the implementation of optimal high frame rate strategies. Beamforming, displacement estimation, and strain estimation parameters were different between the three platforms.

Beamforming was performed internally with the Acuson. Raw RF data obtained with the Vantage and Viewmate Z was beamformed by delay-and-sum. For data obtained with the Viewmate Z and Vantage, axial displacements were calculated from beamformed RF signals using a 1-D normalized cross-correlation kernel. Due to its lower achievable acquisition frame rate, axial displacements were estimated from the beamformed envelope (as opposed to RF) signals on data obtained from the Acuson. For the canine imaging protocol, the displacements observed in the LV and RV were accumulated throughout LV systole, and RV systole, respectively. In the human protocol, the displacements were accumulated during atrial filling, a segment of the cardiac cycle during which the CTI lengthens.

Different phases of the cardiac cycle were imaged in the animal and human models to preserve the directionality of the strain being observed (e.g., the positive strain was estimated in both animal and human models). Axial strains were derived from cumulative axial displacements with a 1-D least-squares estimator (LSQSE). Strain images were smoothed using a 2-D median filter. The specific processing parameters used with each hardware platform are summarized in Table I.

TABLE I
Ultrasound platform processing parameters

| | Probe | | Disp. | Strain | 2D median |
|---|---|---|---|---|---|
| | center | Disp. | kernel | (LSQSE) | filter |
| Ultrasound | freq. | kernel | overlap | kernel | kernel |
| platform | (MHz) | (mm) | (%) | (min) | (mm, °) |
| Verasonics | 5.2 | 3.9 | 90 | 4.3 | (2.1, 2.5) |
| Vantage | | | | | |
| Siemens | 6.0 | 4.8 | 90 | 3.0 | (4.6, 2.9) |
| SC2000 | | | | | |
| Acuson | | | | | |
| Abbott | 6.0 | 1.0 | 90 | 2.3 | (1.4, 2.3) |
| Viewmate | | | | | |
| Z | | | | | |

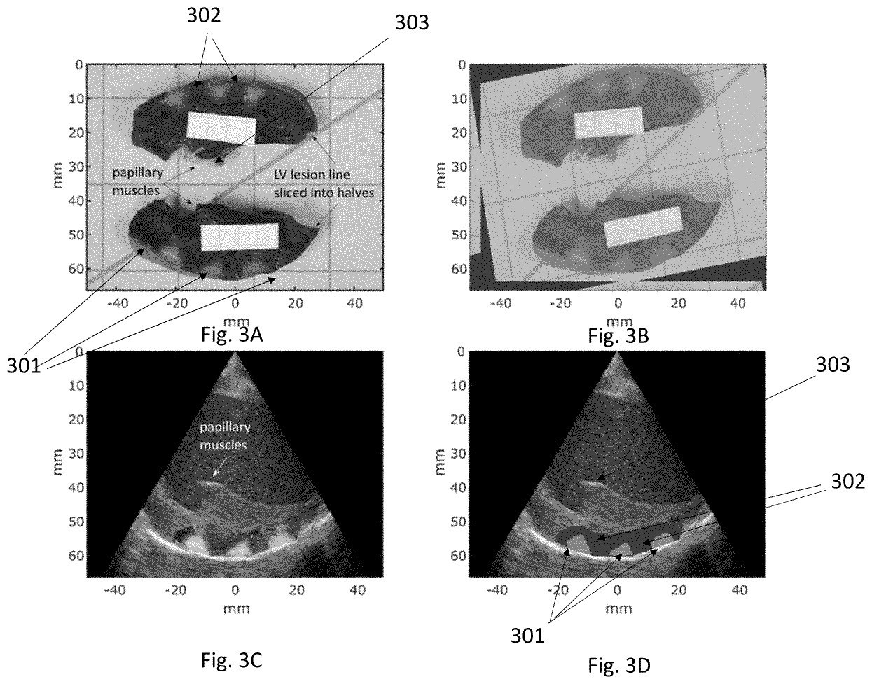

Canine strain images were validated against gross pathology. The images of the TTC-stained and excised lesion lines were converted to grayscale, scaled, aligned with ICE, and overlaid onto the B-mode image based on anatomical landmarks (FIG. 3). TTC stains the lesions white 301; in grayscale, the lesions 301 were brighter compared to non-ablated myocardium. The lesions were masked by a combination of brightness thresholding and manual segmentation (FIG. 3). FIG. 3 provides an exemplary coregistration procedure to validate IME lesion maps with gross pathology in canines. The lesion line was excised from the myocardium after sacrifice and sliced transmurally along the axis of the lesion line to provide a cross-sectional view of the lesion and gap area 302 (FIG. 3A). Using anatomical markers (e.g., papillary muscles 303 and epicardial surface), the intensity image was manually rotated and translated (FIG. 3B) to coregister with the B-mode image (FIG. 3C). Intensity thresholding and manual segmentation were employed to create a binary mask that indicated the lesion area 301 by gross pathology (FIG. 3D).

Due to the translation and deformation of the myocardium during the cardiac cycle, the lesion line can move out of the field-of-view at certain time points during systole. This drop-out was evident upon examination of the strain movie through the entirety of systole. Thus, the number of displacement frames accumulated varied for each acquisition. The strain magnitude was dependent on the number of displacement frames accumulated. The strain image dynamic range was adjusted accordingly in order to maintain a high image contrast between unablated and ablated tissue. The upper bound of the dynamic range (DRupper) was empirically chosen to be half of the median strain at baseline at the number of frames accumulated, as shown in (1).

In (1), ε_{baseline}(n) is the masked strain values in the n^{th} accumulated frame at baseline. The lower bound of the dynamic range was set to 0%. The lesion area estimated by IME was calculated using (2). In (2), ε(i) represents strain at a given pixel i within a masked region consisting of N pixels. εₜₕᵣₑₛₕ is the strain threshold, and Aₚᵢₓₑₗ is the area of each pixel in mm. Masks were manually delineated to isolate lesions (n=10) and gaps (n=6). The boundary of the gap masks was set by a vector spanning the apex of the two lesions, at the points closest to the endocardial wall. The other borders consisted of the lesion perimeters and the epicardial wall. The absolute (δA) and the relative difference between Aₛₜᵣₐᵢₙ and the areas reported by gross pathology (A_{gross}) were calculated using (3). In (3), the brightness threshold pixelₜₕᵣₑₛₕ was determined empirically based on the qualitative assessment of the gross pathology images. Aₚᵢₓₑₗ is the area of each pixel in mm. Manual segmentation was performed to isolate individual lesion areas.

The threshold representing a lesion point, εₜₕᵣₑₛₕ, was empirically derived. The Dice similarity coefficient (DSC) ranges from 0 to 1, and shows the similarity between two binary sets. For example, a DSC of 1 indicates that the binary masks are identical, while a score of 0 indicates that there is no intersection between the positive values in the binary masks. The DSC can be employed in medical imaging analysis (e.g., to compare manual segmentation against an automated method). In terms of true positives (TP), false positives (FP), and false negatives (FN), the DSC between two binary sets A and B can be calculated using (4). In (5), TP corresponds to the tissue that was ablated and correctly identified as such by IME. FP corresponds to regions of unablated tissue that was incorrectly identified as ablated, and FN corresponds to ablated tissue that was incorrectly identified as unablated.

The DSC of the lesion maps produced by strain imaging and gross pathology (the ground truth) were compared for a range of £thresh using (4). In (4), a can be within the range between about 0 and about 1.5. The a value that yields the optimal εₜₕᵣₑₛₕ was determined by calculating the mean DSC curve across the four lesion lines. The a value corresponding to the maximum of the mean DSC was chosen to calculate εₜₕᵣₑₛₕ in the canine model.

For human models, the CTI was manually segmented. The median axial strain was calculated within the CTI for the five patients. Median strain (£median) at the CTI at baseline and at the conclusion of the procedure (once the block was achieved) was statistically compared via the Student's paired t-test. In contrast to the canine protocol, no thresholding was performed to isolate individual lesions, and the dynamic range was set to [−40%, 40%] for all cases.

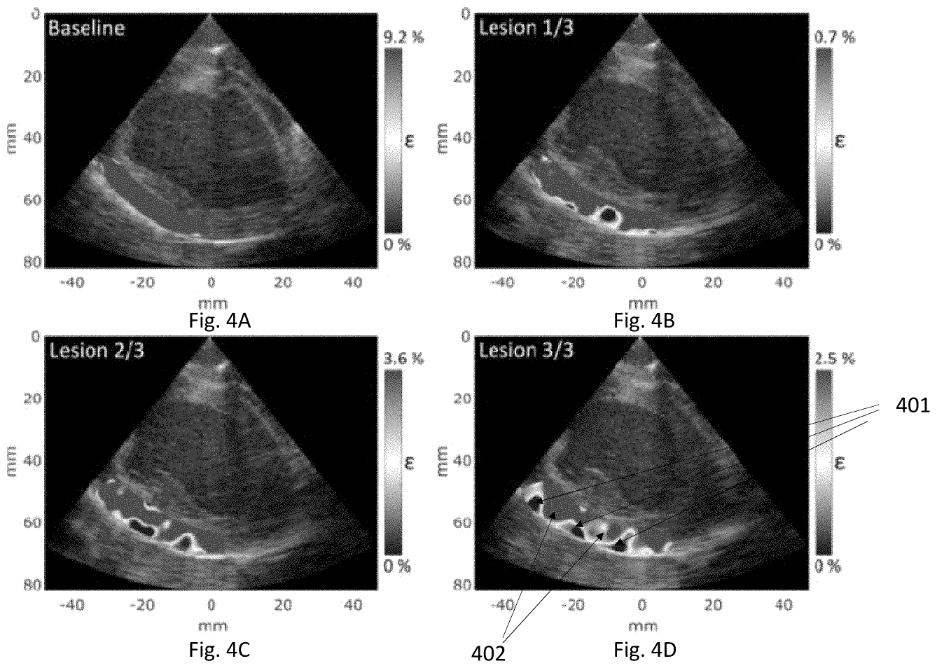

Open chest canine ablation: IME was capable of accurately capturing the formation of the LV lesion line throughout the ablation procedure (FIG. 4). At baseline, LV strain was homogenously positive and high magnitude (ε≥DRᵤₚₚₑᵣ) throughout (FIG. 4A). In contrast, lesions manifested as regions of low strain

$$( ɛ < \frac{D {R}_{u pper}}{2} ) .$$

The strain images demonstrate the progression of lesion line formation, lesion-by-lesion (FIG. 4B-4D). At the conclusion of the ablation experiment, three distinct lesions 401 and two distinct gaps 402 are visible (FIG. 4D).

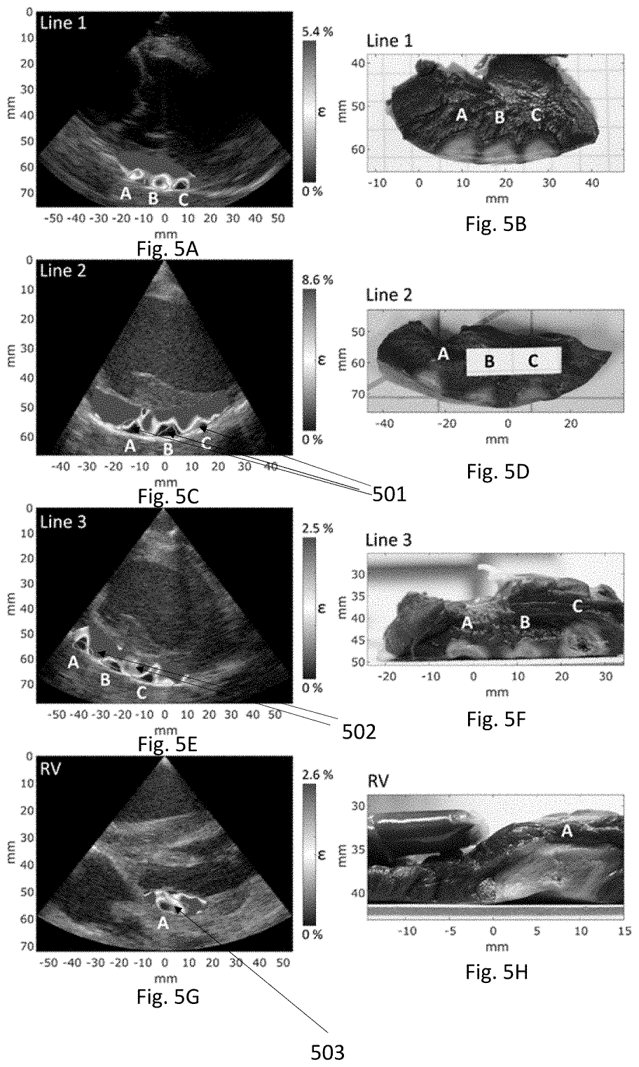

The contours of the lesion areas, as indicated by the TTC-stained tissue sections were overlaid onto the post-ablation strain-based lesion maps (FIGS. 5A-5H). Qualitatively, there is good agreement between the lesions detected by IME and the gross pathology. In each of the three LV lesion lines, IME correctly identified three lesions 501 and two gaps 502. The singular lesion 503 in the RV was also correctly identified.

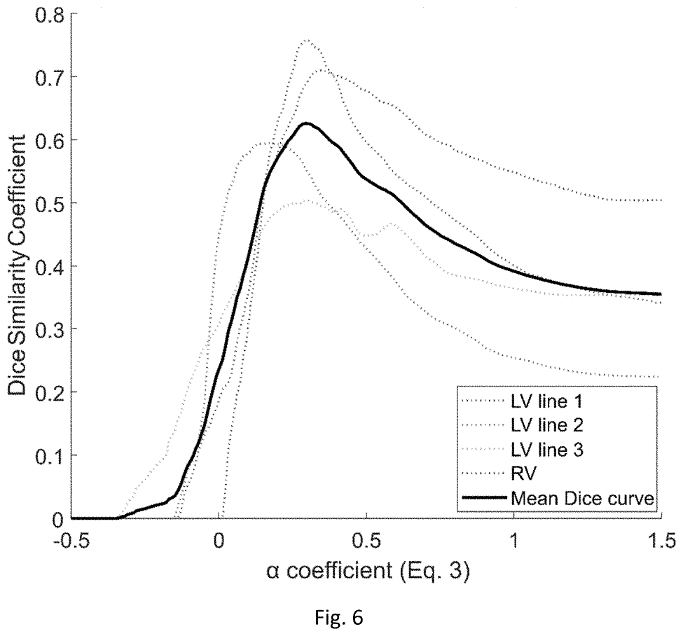

Calculating the DSC across a range of a values yielded the plot summarized in FIG. 6. The maximum of the mean DSC curve was found at α=0.27, wherein a DSC value of 0.62 was found.

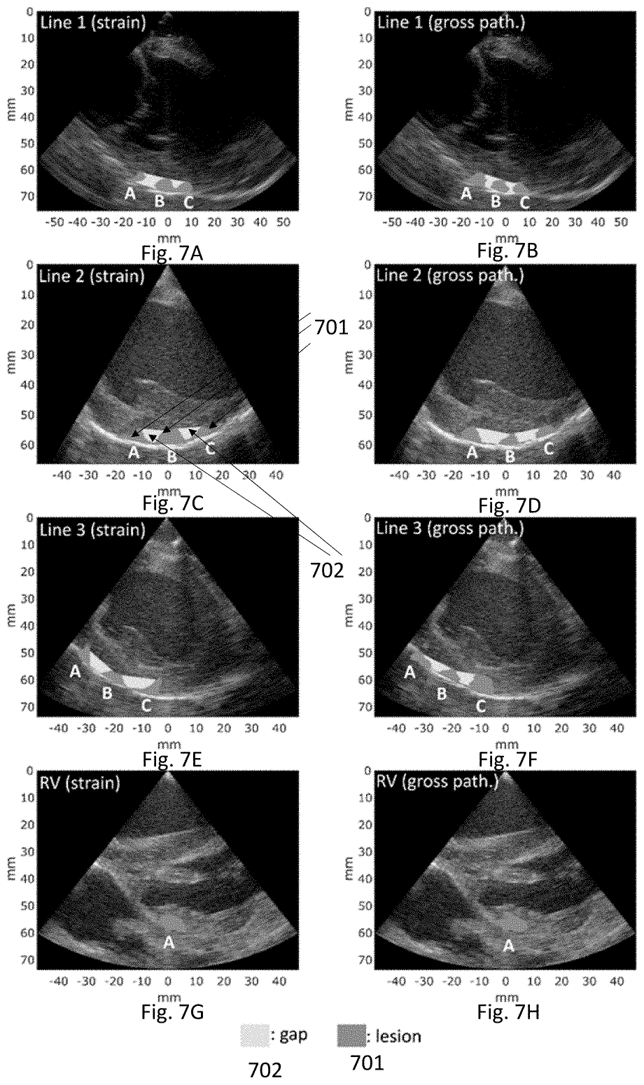

The lesion and gap areas, as determined by IME versus gross pathology, are summarized in FIG. 7. The lesion areas 701 are designated in turquoise, with the gaps 702. Qualitatively, the thresholded strain lesion maps compare well against the gross pathology. The lesion areas found by strain and gross pathology are summarized and compared in Table II.

TABLE II
Lesion area by IME strain and gross pathology in the canine

| | Aₛₜᵣₐᵢₙ | A_{gross} | δA | Relative |
|---|---|---|---|---|
| Lesion | (mm²) | (mm²) | (mm²) | error (%) |
| Line 1, Lesion A | 28 | 15 | 13 | 46 |
| Line 1, Lesion B | 27 | 34 | 7.9 | 30 |
| Line 1, Lesion C | 18 | 23 | 4.8 | 26 |
| Line 2, Lesion A | 30 | 28 | 2.1 | 7.0 |
| Line 2, Lesion B | 20 | 45 | 24 | 120 |
| Line 2, Lesion C | 21 | 22 | 0.82 | 3.9 |
| Line 3, Lesion A | 29 | 28 | 1.1 | 3.9 |
| Line 3, Lesion B | 26 | 32 | 6.8 | 26 |
| Line 3, Lesion C | 65 | 42 | 23 | 35 |
| Line 4, Lesion A | 72 | 62 | 9.5 | 13 |
| Mean | 34 | 33 | 9.3 | 31 |
| STD | 19 | 14 | 8.4 | 34 |

Gap area assessment is summarized in Table III.

TABLE III
Gap area by IME strain and gross pathology in the canine

| | A_{gross} | Aₛₜᵣₐᵢₙ | δA | Relative |
|---|---|---|---|---|
| Gap | (mm²) | (mm²) | (mm²) | error (%) |
| Line 1, Gap AB | 19 | 32 | 13 | 66 |
| Line 1, Gap BC | 11 | 11 | 0.63 | 5.5 |
| Line 2, Gap AB | 43 | 25 | 18 | 43 |
| Line 2, Gap BC | 25 | 22 | 3.2 | 13 |
| Line 3, Gap AB | 29 | 39 | 10 | 35 |
| Line 3, Gap BB | 30 | 53 | 23 | 78 |
| Mean | 26 | 30 | 11 | 40 |
| STD | 11 | 15 | 9 | 29 |

By gross pathology, the lesion and gap areas (A_{gross}) measured 34±19 mm² and 26±11 mm² on average, respectively. By IME strain imaging, the lesion areas and gaps (Aₛₜᵣₐᵢₙ) were estimated to be 33±14 mm² and 30±15 mm² on average, respectively.

The individual difference in lesion area between strain and gross pathology (δA) ranged 0.82-24 mm², with a mean difference of 9.3±8.4 mm². In terms of relative error, the difference ranged from 3.9 to 120%, with a mean relative difference of 31±34%.

The individual difference in gap areas between strain and gross pathology ranged from 0.63 to 23 mm², with a mean difference of 11±9.0 mm². In relative error, the difference ranged 5.5-78%, with a mean relative difference of 40±29%.

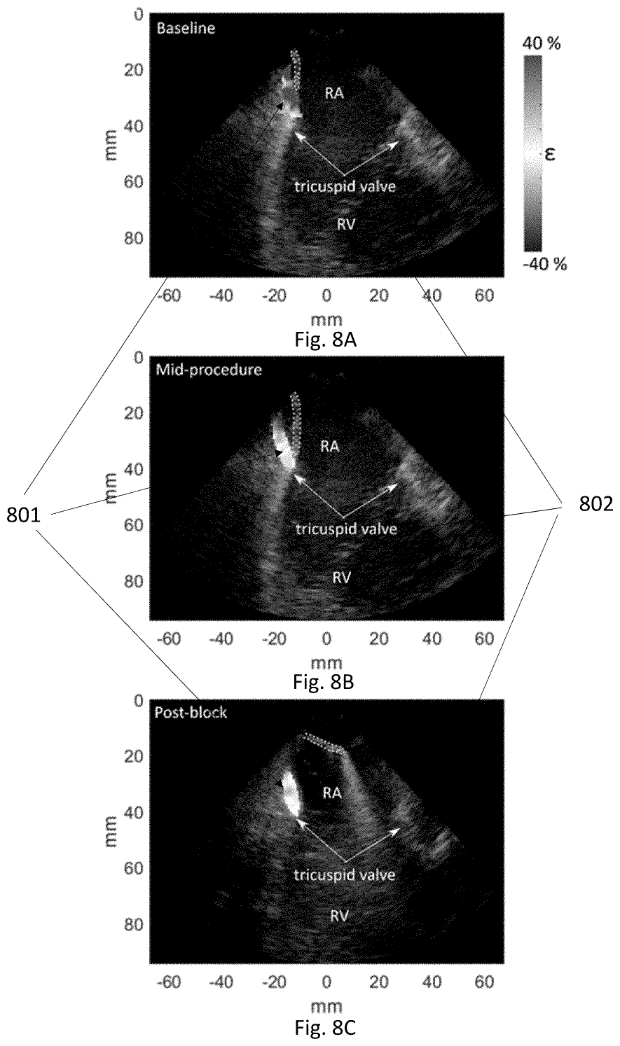

Atrial flutter CTI ablation: lesion mapping in a patient receiving CTI ablation to relieve atrial flutter is demonstrated in FIG. 8. Images were acquired at baseline (FIG. 8A), during (FIG. 8B), and after (FIG. 8C) CTI ablation. The lesion line 801 was initiated proximal to the tricuspid valve 802 and progressed towards the direction distal the tricuspid valve during the ablation. At baseline, the CTI exhibits homogenously positive, high-magnitude strain (i.e., strain greater than about 20%) (FIG. 8A). During the procedure, strain in the region proximal the tricuspid valve is low magnitude (i.e., strain less than about 20%), while the region distal the valve (thus far in the procedure unablated) still possesses high-magnitude positive strain (FIG. 8B). Finally, the entire CTI is observed to have a low-magnitude strain at the conclusion of the ablation procedure (FIG. 8C).

In all five patients imaged, ε_{median} in the CTI decreased after ablation compared to baseline. The mean paired difference in CTI strain was −17±11%. Employing a two-sided paired t-test, the difference was determined to be statistically significant (p<0.05).

The potential of IME for lesion and gap visualization and quantification with intracardiac echocardiography was investigated. Employing an open-chest canine model, IME was capable of resolving all ten lesions and all six lesion gaps generated in three LVs and one RV. IME was then used to track atrial flutter ablation in human subjects. There was a reduction in strain in the ablated region (the CTI) in all five patients after the ablation procedure.

RF ablation generates lesions that are mechanically non-compliant and stiffer compared to unablated tissue. Since non-compliant and stiff tissue hardly deforms, the magnitude of the strain difference between ablated and unablated tissue can be significant. This reduction in strain following ablation can be the mechanism that allows IME to image the lesion line with high contrast resolution.

The strain magnitude within the ablated area was significantly reduced compared to unablated tissue in the canine. The dynamic range was chosen to increase the visual contrast between scar and unablated tissue; healthy tissue manifests as regions of high magnitude positive strain while scarred tissue manifests are regions of low magnitude strain (FIG. 4). These low-magnitude strain regions were indicative of non-contractile scar tissue, verified by overlaying the ground truth lesion contours as defined by gross pathology (FIGS. 5A-5H). Qualitatively, there was excellent agreement between the IME lesion maps and the gross pathology. IME correctly represented the three lesions in each canine LV as a non-contiguous linear line (FIGS. 5A-5F).

Thresholding was employed to allow for quantitative comparison of lesion and gap area between strain imaging and gross pathology (Tables II and III). The strain threshold under which a region of tissue can be classified as the scar was chosen based on the peak mean Dice curve (FIG. 6).

Thresholding can also be a useful tool for visualization, simplifying the interpretation of the strain images. Given the number of variables that can affect strain magnitude (such as preexisting ischemia or infarct, the orientation of the heart wall in the field-of-view), setting a hard threshold based on strain magnitude can be challenging. Since the optimal threshold can be variable among patients, imaging conditions, and the heart chamber being imaged, the threshold can be an adjustable parameter. This adaptive threshold can be based on the baseline strain of a given patient and can be dynamically set based on the experience of the electrophysiologist. Furthermore, the thresholded image is not intended to be a substitute for the strain image. In the same way, speckle-tracking echocardiography can be used. IME's strain images can become interpretable as electrophysiologists gain experience with the technology.

Gap resolution was improved compared to existing implementations of IME. In certain IME techniques, the smallest detectable gap measured from lesion edges at the epicardial level can be about 15 mm. The disclosed subject matter can provide IME techniques capable of resolving gaps as small as 11 mm², or 3.6 mm measured edge-to-edge at the epicardial level (Table III and FIG. 7A-7B).

The improved lesion mapping is due to the implementation of superior high frame-rate transmit strategies. Instead of a single-diverging wave sequence, a 15-source compounding (Verasonics) and 23-source composite plane wave sequence (Siemens) were applied in the canine model. Increased gap resolution improvement can be attributable to improved SNR and lateral resolution over single-source diverging wave imaging.

Certain methods to best align the lesion line and ultrasound plane in the canine model was performed. In conjunction with SOUNDSTAR, the Acuson was able to graphically mark the location of the ablation catheter when it was in-plane. In the acquisitions taken with the Vantage, each potential location was manually palpated prior to ablation; if the location was in-plane, the resulting tissue deformation would be evident in the B-mode. Nonetheless, an inherent source of error can be that a 2-D strain image of a live tissue target featuring 3-D translation and deformation was compared against a 2-D section of gross pathology. A lower degree of agreement in the lesion and gap areas can be attributable to the imperfect coregistration of the two 2-D representations of the lesion line. This imperfect coregistration likely inflated the error of the lesion and gap area estimation, 31±34% and 40±29%, respectively (Tables II and IOI), and led to a relatively low maximum DSC of 0.62. The addition of contrast-enhanced Magnetic Resonance Imaging can mitigate this source of error. The combination of MRI 3-D visualization of lesion line and gross pathology can allow for reliable assessments of lesion and gap area accuracy. Furthermore, the combination identifies how much error is attributable to poor coregistration versus the resolution limitations of IME.

The feasibility of IME in intracardiac echocardiography as a useful diagnostic tool was assessed by comparing the strain in the CTI before and after atrial flutter ablation. The strain was calculated during the atrial filling of the RV, during which the CTI exhibits extension (i.e., stretches). Scar tissue is stiffer and less compliant; ablated regions of the atrial wall exhibit significantly reduced extension during atrial filling, manifesting as regions of low-magnitude strain (<20%) in IME lesion maps. The progression of a CTI ablation is summarized in FIG. 8. The ablation catheter begins proximal the valve and progresses along with the CTI in the direction distal the valve, the location of previous lesions clearly indicated by the strain drop compared to the baseline. IME was able to differentiate between the CTI before and after ablation (mean Δε=−17±11%), with every patient recording a decrease in ε_{median} at the end of the procedure.

IME was able to be integrated into the current ablation workflow with minimal adaptations, especially since the use of ICE is the standard of care in many cardiac ablation procedures. Certain techniques for imaging the lesion line require additional hardware, such as MRI, or an additional probe to induce a push beam. In contrast, IME was integrated into an ICE platform that was already being used in ablation procedures at the clinic. IME was implemented in three different hardware platforms employing different high-frame-rate sequences, a testament to the flexibility and adaptability of this technique. Furthermore, IME can provide an improved field-of-view of the lesion line in contrast to photoacoustic or acoustic radiation force impulse (ARFI) methods, allowing for quicker assessment of an ablation procedure's progress.

The displacement estimation algorithm depended on the hardware platform that was used to collect the acquisitions. Displacement estimation was performed using 1-D cross-correlation on the RF data derived from the Verasonics Vantage and Abbott Viewmate Z. In contrast, 1-D cross-correlation was performed on the envelope of the RF signal on data derived from the Siemens Acuson system. RF-based motion estimators are considered more accurate than envelope-based estimators at high frame rates since the former contains phase information. However, RF-based estimators can perform poorly if the acquisition frame rate is too low, or a large window size is employed, due to decorrelation from false peak or jitter errors. The Siemens Acuson transmit sequence frame rate used herein (≤250 Hz) was substantially lower than that of the Verasonics (460 Hz) or Abbott (600 Hz) systems. Envelope-based displacement estimation was necessary as limitations in the programmability of the Acuson to prevent imaging at higher frame rates. The envelope-based estimator can be preferred for the Siemens data, given the frame rate limitations. While displacement estimation on RF versus beamformed envelope signals is comparable in canine models, the latter approach can require a larger displacement window size that can compromise the strain resolution.

1-D cross-correlation of the envelope signal was performed instead of using a 2-D kernel. A 1-D kernel can be more accurate due to the edge mismatch between adjacent plane waves in the transmit sequence, visible in the B-mode (FIGS. 3C and 3D). While this mismatch in the lateral direction can lead to substantial decorrelation when employing a 2-D kernel, this artifact is not relevant when using a uniaxial estimator.

The implementation of IME described herein estimated 1-D (axial) motion. In both the canine and human study, ultrasound views were chosen such that the predominant direction of myocardial motion was in the axial direction. In clinical settings, 2-D or 3-D motion can be estimated to circumvent angle dependence in the strain calculation.

The disclosed IME can be integrated with an electroanatomic system. The clinical model can be improved by using the disclosed software, which is capable of visually tagging the ablation catheter leading to better alignment with the lesion line. Linking IME to an electroanatomic system can allow for the generation of stain lesion maps that can be registered to specific positions in the myocardium, allowing for a pseudo-3D visualization. Alternatively, a 3D ICE catheter can be employed.

The disclosed subject matter provides IME techniques to visualize the lesion line and inform ablation procedures. Gap resolution of IME lesion mapping was validated in an open-chest canine model that tracked epicardial ablations in the ventricles, with the smallest gap tested being 11 mm2 (3.6 mm on the epicardial surface). A clinical feasibility assessment was performed to demonstrate the diagnostic utility of strain and to show that IME can be integrated into ablation procedures with minimal modifications to the current workflow. Additional feasibility in animals and humans are warranted to prove that IME is a viable ablation monitoring approach for atrial arrhythmias.

The features, structures, or characteristics of certain embodiments described throughout this specification can be combined in any suitable manner in one or more embodiments.

One having ordinary skill in the art will readily understand that the disclosed subject matter as discussed above can be practiced with procedures in a different order, and/or with hardware elements in configurations which are different than those which are disclosed. Therefore, although the disclosed subject matter has been described based upon these embodiments, it would be apparent to those of skill in the art that certain modifications, variations, and alternative constructions would be apparent while remaining within the spirit and scope of the disclosed subject matter.

What is claimed is:

1. A system for monitoring cardiac ablation procedures, comprising:
an imaging device configured to acquire successive frames and radio frequency signal data of a heart; and
an image processor, coupled to the imaging device, configured to:
obtain a signal envelope of the radio frequency signal data;
generate a strain map based on the signal envelope;
apply a strain threshold to the strain map for classification of lesion tissue;
provide an image that visualizes lesion formation during the cardiac ablation procedures in real-time.

2. The system of claim 1, wherein the radio frequency signal data is obtained using an acquisition frame rate of less than 500 frames per second (fps).

3. The system of claim 1, wherein an axial, incremental, or cumulative axial strain is estimated using the signal envelope

4. The system of claim 3, wherein the strain map is generated based on the axial, incremental, and/or cumulative axial strain of the successive frames.

5. The system of claim 1, further comprising a catheter for the cardiac ablation procedures.

6. The system of claim 1, wherein the radio frequency data is acquired using intracardiac echocardiography.

7. The system of claim 5, wherein the catheter is used for real-time imaging through intracardiac echocardiology.

8. The system of claim 1, wherein the lesion formation is represented as a near-zero magnitude strain.

9. The system of claim 1, wherein the lesion formation is represented as a relatively lower magnitude strain.

10. The system of claim 4, wherein the axial, incremental, and/or cumulative strain is estimated based on axial, incremental, and/or cumulative axial displacement of the heart.

11. The system of claim 10, wherein the axial, incremental, and/or cumulative axial displacement of the heart is estimated by performing a cross-correlation on the signal envelop.

12. The system of claim 1, wherein a lateral, incremental lateral, or cumulative lateral strain is estimated based on lateral, incremental, and/or cumulative lateral displacement of the successive frames.

13. A method for monitoring cardiac ablation procedures, comprising:
obtaining a signal envelope of a radio frequency signal data;
generating a strain map based on the signal envelope;
applying a strain threshold to the strain map for classification of lesion tissue; and
providing an image that visualizes lesion formation during the cardiac ablation procedures in real-time.

14. The method of claim 13, wherein the radio frequency signal data is obtained using an acquisition frame rate less than 500 frames per second (fps).

15. The method of claim 13, wherein the strain map is generated based on axial, incremental, or cumulative axial strain of the successive frames.

16. The method of claim 15, wherein the axial, incremental, or cumulative axial strain is estimated using the signal envelope.

17. The method of claim 13, wherein the radio frequency data is acquired using intracardiac echocardiography.

18. The method of claim 17, wherein a catheter is used for real-time imaging through the intracardiac echocardiology.

19. The method of claim 13, wherein the lesion formation is represented as near-zero magnitude strain.

20. The method of claim 16, further comprising performing a cross-correlation on the signal envelope.

21. The method of claim 15, wherein the axial, incremental, or cumulative axial strain is estimated based on axial, incremental, or cumulative axial displacement of the successive frames.

22. The method of claim 13 further comprising estimating a lateral, incremental lateral, or cumulative lateral strain based on lateral, incremental, and/or cumulative lateral displacement of the successive frames.
